(19) United States
(12) Patent Application Publication
(10) Pub. No.: US 2002/0193321 A1
(43) Pub. Date: Dec. 19, 2002

(54) Dual dispenser for aesthitically acceptable delivery of anhydrous skin treatment compositions

(76) Inventors: Mohan Vishnupad, Easton, CT (US); Naomi Vishnupad, Easton, CT (US)

Correspondence Address: Peter Deluca, Carter, DeLuca, Farrell & Schmidt, LLP, Suite 225, 445 Broad Hollow Road, Melville, NY, 11747, US

(21) Appl. No.: 10/151,417

(22) Filed: May 20, 2002

Related U.S. Application Data
(63) Continuation-in-part of application No. 10/121,839, filed on Apr. 12, 2002.
Application No. 10/121,839 is a continuation-in-part of application No. 09/734,748, filed on Dec. 12, 2000, now Pat. No. 6,462,025.

(51) Int. Cl. A61K 31/7048; A61K 31/65; A61M 5/00; A61K 31/375; A61K 35/78

(52) U.S. Cl. 514/29; 514/152; 424/729; 424/757; 514/474; 604/233

(57) ABSTRACT

Two separate compositions, one containing multiple doses of an anhydrous first composition that includes a first active ingredient that is hydrolytically unstable and which provides a benefit to the skin of a user; and a second aqueous composition which may or may not contain a second active ingredient. are packaged within and dispensed from a common dispenser. By packaging these two compositions in this manner, long shelflife, good aesthetics and convenient dispensing and application are provided.

### RELATED APPLICATIONS

This application is a continuation-in-part of U.S. application Ser. No. 09/734,748 filed on Dec. 12, 2000 the disclosure of which is incorporated herein in its entirety by this reference.

### BACKGROUND

1. Technical Field

This disclosure relates to compositions and apparatus for dispensing two distinct substances. More specifically, this disclosure relates to compositions and apparatus which allow long-term storage and subsequent dispensing of two compositions, to wit, an anhydrous first composition containing a first active ingredient for treating skin, the first active ingredient being unstable in the presence of water and an aqueous second composition.

2. Background of Related Art

Many skin treatment active ingredients are extremely sensitive to water and easily breakdown resulting in an unstable product with reduced or total loss of potency. Vitamin A, vitamin C, hydroquinone and dihydroxyacetone are examples of skin treatment active ingredients that are quite unstable in aqueous media and therefore not available in cosmetic formulations for consumer use. These active ingredients by themselves or in combination have beneficial effect in treating facial wrinkles and dry skin. For example, Vitamin C and hydroquinone in combination provide good skin lightening benefits. However, in aqueous media Vitamin C discolors and breaks down completely.

Anhydrous compositions for hydrolytically unstable compounds have been proposed. However, such anhydrous compositions are normally commercially unacceptable due to poor aesthetics or poor feel to the end user.

It would be desirable to provide a means for storing and dispensing active skin treating compounds in an anhydrous composition (which allows prolonged shelf life for the active) and which provides an aesthetically acceptable product for application to the skin.

### SUMMARY

It has now been discovered that two separate compositions, one being an anhydrous composition containing a first active ingredient which is unstable in the presence of water and which provides a beneficial effect to skin and one being a water-containing composition that may or may not contain an active ingredient, can be packaged within and dispensed from a common dispenser. More particularly, a dual dispenser and has two chambers and one or more outlets for dispensing first and second compositions from the chambers. The first chamber contains multiple doses of an anhydrous first composition that includes a first active ingredient that is hydrolytically unstable and which provides a benefit to the skin of a user; and the second chamber contains an aqueous second composition which may or may not contain a second active ingredient. The term “hydrolytically unstable” as used herein means that the active ingredient changes chemically in the presence of water to a form that has either reduced potency or total loss of potency compared to the original active ingredient. By packaging the two compositions in this manner, long shelflife is achieved for the water-sensitive active ingredient and convenient dispensing and application of an aesthetically acceptable product are provided.

In one particularly useful embodiment, a dual dispenser contains i) multiple doses of an anhydrous first composition that includes a polar solvent, a water-sensitive active ingredient and a thickening agent; and ii) multiple doses of a second composition selected from the group consisting of aqueous solutions, aqueous suspensions, oil-in-water emulsions and water-in-oil emulsions.

The major benefits of such dual dispensing systems (such as, for example, dual pump, dual portioned tubes, etc.) are:

1. Such package keeps the active ingredients which as are water sensitive, separate from aqueous media.

2. The aqueous composition will provide necessary hydration which is normally important to consumers for any skin application.

3. The anhydrous polar gels system are quite sticky and aesthetically not acceptable for cosmetic use. Blending with aqueous composition at the time of use overcomes any aesthetic negatives and provides consumer-acceptable cosmetic skin treatment product.

4. Anhydrous composition can contain one or more active ingredients such as Vitamin A., Vitamin C, hydroquinone in one phase of the dual dispensing package.

5. Dual dispensing package also allows us to deliver active ingredients from anhydrous composition and active ingredients which are stable in aqueous system from second composition for maximum skin benefits.

### BRIEF DESCRIPTION OF THE DRAWINGS

Various embodiments are described herein with reference to the drawing wherein:

FIG. 1 is a schematic view of a container suitable for dispensing the first and second compositions in accordance with this disclosure.

### DETAILED DESCRIPTION OF THE PREFERRED EMBODIMENT

The dual dispensers described herein include a first chamber containing a first composition, a second chamber containing a second composition and one or more outlets for simultaneously dispensing the first and second compositions.

The first and second compositions preferably have a viscosity greater than 1000 centipoise (cps) when measured using a Brookfield viscometer (model LVT) at room temperature using spindle number 3 or 4 at 0.3 to 30 rpm. It should be understood that all viscosities referred to herein are measured in this manner. Preferably, the first and second compositions have a viscosity greater than 5,000 cps. In particularly useful embodiments, the compositions have a viscosity in the range of from about 1000 to about two million centipoise. Most preferably, the first and second compositions have a viscosity in the range of about 10,000 cps to about 1,000,000 cps. For purposes of presenting a composition with a good feel to the user, the first and second compositions advantageously have viscosities that differ by no greater than 25%.

The first composition is substantially anhydrous and contains a first active ingredient that is susceptible to deterioration from contact with water and provides a benefit to the skin of a user. By the term “substantially anhydrous” it is meant that, other than water of hydration contained in the various components used to formulate the composition, no free water is added to the composition. Typically, the water content of the composition will be less than 5% by weight. Preferably the water content of the composition is less than 3% and most preferably less than about 1% by weight of the composition.

Suitable active ingredients that can be incorporated in the anhydrous first composition include, but are not limited to antibiotics, ascorbic acid, ascorbic acid derivatives, retinoids, hydroquinone, dihydroxyacetone, licorice extract and green tea extract.

One class of active ingredients known to provide a beneficial effect to the skin of a user is antibiotics. Preferably the antibiotic is one currently known to be useful in treating acne, such as, for example, erythromycin, tetracyclin, clindamycin, their derivatives or pharmaceutically acceptable salts. The antibiotic is present in the first composition in an effective acne-treating amount, preferably an amount from about 0.001 wt. % to about 5 wt. %, more preferably about 0.1 wt. % to about 1.0 wt. %.

In a particularly useful embodiment, the first composition is substantially anhydrous and contains a polar solvent, a thickening agent and an antibiotic.

Polar solvents useful in this embodiment of the first composition include polyols. A polyol is a compound with at least two hydroxyl groups per molecule, i.e., a compound having multiple hydroxyl groups as part of its molecular structure. Among the useful polyols are polyhydric alcohols. Propylene glycol, dipropylene glycol, polyethylene glycol and glycerine are particularly preferred polar solvents for use in the first composition.

Any thickening agent capable of imparting a desired viscosity to an anhydrous composition can be used in this embodiment. Suitable thickening agents include but are not limited to acrylic acid polymers and polyacrylamides. The thickening agent are used in an amount sufficient to obtain a composition of viscosity in the desired range. The specific amount of thickener employed will depend on a number of factors including the solvent used and the desired viscosity to be achieved. The thickener is present in the first composition at a level from about 0.05% to about 20%, preferably from about 0.5% to 10% and most preferably from about 1% to about 10%.

In an alternative embodiment, the first composition contains a retinoid. Suitable retinoids, include, for example, retinol, retinoic acid, retinyl palmitate, retinyl propionate or retinyl acetate as well as synthetic retinoid mimics. The retinoid is preferably present in the second composition in an amount from about 0.001 wt. % to about 5 wt. %, more preferably about 0.1 wt. % to about 2.0 wt. %.

In a particularly useful embodiments, the retinoid-containing compositions are also substantially anhydrous and contains a polar solvent, a thickening agent and a retinoid. Suitable polar solvents and thickening agents for the second composition are the same as described above for the antibiotic compositions described above. In this alternative embodiment, the retinoid-containing composition can have a viscosity greater than about 1000 centipoise (cps) when measured using a Brookfield viscometer (model LVT) at room temperature using spindle number 3 or 4 at 0.3 to 30 rpm. Preferably, the retinoid-containing composition has a viscosity greater than 5,000 cps. In particularly useful embodiments, the retinoid-containing composition has a viscosity in the range of from about 1000 to about two million centipoise. Most preferably, the retinoid-containing composition has a viscosity in the range of about 10,000 cps to about 1,000,000 cps.

In an alternative embodiment, the first composition contains an ascorbic acid compound (i.e., ascorbic acid (vitamin C) or its salts, ascorbyl esters of fatty acids, ascorbic acid derivatives, such as, for example, magnesium ascorbyl phosphate. The ascorbic acid compound is preferably present in the composition in an amount from about 0.001 wt. % to about 15 wt. %, more preferably about 0.1 wt. % to about 5.0 wt. %.

In a particularly useful embodiments, the ascorbic acid compound-containing compositions are also substantially anhydrous and contains a polar solvent, a thickening agent and an ascorbic acid compound. Suitable polar solvents and thickening agents for the second composition are the same as described above for the antibiotic and retinoid compositions described above. In this alternative embodiment, the ascorbic acid compound-containing composition can have a viscosity greater than about 1000 centipoise (cps) when measured using a Brookfield viscometer (model LVT) at room temperature using spindle number 3 or 4 at 0.3 to 30 rpm. Preferably, the ascorbic acid compound-containing composition has a viscosity greater than 5,000 cps. In particularly useful embodiments, the ascorbic acid compound-containing composition has a viscosity in the range of from about 1000 to about two million centipoise. Most preferably, the ascorbic acid compound-containing composition has a viscosity in the range of about 10,000 cps to about 1,000,000 cps.

The second composition contains water and is maintained in a separate chamber from the first composition to avoid any adverse effect on the first active ingredient. The second composition can be any aqueous formulation including solutions, suspensions, oil-in-water emulsions and water-in-oil emulsions. The second composition provides the hydration necessary to release the active ingredients in the anhydrous composition and make the active ingredient readily available to the skin. Secondly the end product aesthetics can be easily controlled by the formulation of the aqueous emulsions and gel.

The second composition can optionally contain an active ingredient. The second active ingredient may be effective in treating acne or may provide some other beneficial effect upon topical administration to a user's skin (such as, for example, alpha-hydroxy acids or anti-irritants, etc.)

Benzoyl peroxide is one active ingredient known to be an effective anti-acne treatment that can be incorporated into the second composition. The second composition can any benzoyl peroxide-containing cream, lotion, gel or suspension. Benzoyl peroxide compositions that are suitable for use in accordance with this disclosure are readily formulated by those skilled in the art and include, but are not limited to the compositions disclosed in U.S. Pat. Nos. 4,606,913; 4,671,956; 5,019,567; 5,879,716; and 5,998,392 the disclosures of which are incorporated by this reference. The amount of benzoyl peroxide in the composition can be from about 0.1 to about 20 percent by weight based on the total weight of the composition, preferably from about 1.0 to about 15 weight percent, most preferably from about 1.5 to about 10 weight percent. In this embodiment, the benzoyl peroxide-containing composition can have a viscosity greater than about 1000 centipoise (cps) when measured using a Brookfield viscometer (model LVT) at room temperature using spindle number 3 or 4 at 0.3 to 30 rpm. Preferably, the benzoyl peroxide-containing composition has a viscosity greater than 5,000 cps. In particularly useful embodiments, the benzoyl peroxide-containing composition has a viscosity in the range of from about 1000 to about two million centipoise. Most preferably, the benzoyl peroxide-containing composition has a viscosity in the range of about 10,000 cps to about 1,000,000 cps.

The first and second compositions preferably have viscosities that are similar to provide a cosmetically elegant product when the first and second compositions are simultaneously dispensed. In particularly useful embodiments the difference in viscosity between the first and second compositions is no more than about 25%.

In addition to the above-listed ingredients, one or both of the first and second compositions may also contain a variety of non-essential ingredients such as, for example, co-solvents, preservatives, emollients, humectants, skin lightening agents, anti-inflammatory agents, antioxidants, insect repellents or skin cooling compounds, etc. For example, either of the first or second composition may contain one or more co-solvents, such as ethanol, acetone or propylene carbonate. As another example, either of the first or second compositions may contain licorice extract and green tea extract which are examples of skin lighteners that can be used alone or in conjunction with vitamin C, vitamin A and other hydrolytically unstable active ingredients.

A preservative can also be used in either or both of the first or second compositions. Preservatives suitable for use in connection with the present compositions include parabens, sorbates, benzyl alcohol, diazolidinyl urea and isothiazolinones. Preservatives can be present in an amount from about 0.001 wt. % to about 15 wt. % of the total composition.

One or both of the first or second compositions can also be formulated to contain about 0.01 wt. % to about 30 wt. %, preferably about 1.0 wt. % to about 15 wt. % of the total composition, skin cooling compounds, such as menthol, methyl glycerol, asymmetrical carbonates, thiocarbonates and urethanes, substituted carboxamides, ureas or phosphine oxides as described in J. Cosmet. Chem., vol. 29, page 185 (1978) and incorporated herein by reference, methyl lactate and menthone glycerin acetal.

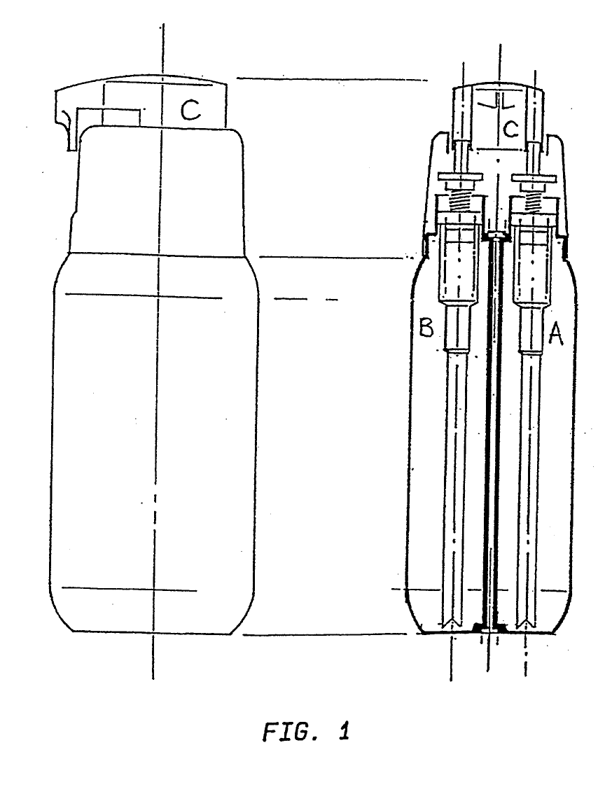

The first substantially and second compositions are stored in and dispensed from a multi-chamber dispenser. Dispensing systems that include pump means suited for simultaneously dosing two separately contained incompatible compounds are well known. As such, the dispensing system schematically depicted in FIG. 1 (dispenser from Maplast, Tradate, Italy) is just one example out of a number of products which range from small, two-chambered single use pouches to tubes using different product compartments or tubes compartmentalized using extrudable, viscous and relatively inert materials to separate the incompatible compounds.

The dispenser shown in FIG. 1 is able to simultaneously dose two compounds separately contained in A and B by pressing dosing head C. Pressing dosing head C activates two small pumps which subsequently dispense the two compounds in approximately equal volumes. Depending on the design of the dosing head, the compounds can be dosed in two separate streams or in just one stream. If desired, a dispensing unit that is able to deliver The first and second substantially anhydrous compositions in a ratio, such as, for example, 1:2 can be used. Translated to the dispenser depicted in FIG. 1, this would mean that one of the two pumps is able to dose at least twice the volume of the other pump in just one stroke of dosing head C. Translated to a two-chambered single use pouch, this would mean that the chamber containing the first substantially anhydrous composition contains at least half as much product volume as the other chamber. Translated to a two-compartment tube, this would mean that under equal pressure the discharge orifice for the compartment containing the first substantially anhydrous composition allows the passage of at least twice as much product as the discharge orifice of the other compartment. Translated to a tube which is compartmentalized using extrudable material, this would mean that first substantially anhydrous composition is present inside the tube in at least double the volume of the second substantially anhydrous composition.

Other suitable dispensers are disclosed in U.S. Pat. Nos. 5,356,040; 5,823,391, and 4,826,048 the disclosures of which are incorporated herein by this reference.

The following examples are presented to illustrate specific embodiments of the present compositions and methods. These examples should not be interpreted as limitations upon the scope of the invention.

### EXAMPLES 1-10

The following formulations are exemplary of substantially anhydrous antibiotic compositions suitable for use as the first composition:

| Example 1 | |
|---|---|
| Erythromycin | 2 |
| Propylene glycol | 96 |
| ULTREZ 10 | 2 |
| Polyethylene glycol | — |
| Clindamycin | — |
| Example 2 | |
| Erythromycin | 2 |
| Propylene glycol | 71.5 |
| ULTREZ 10 | 1.5 |
| Polyethylene glycol | 25.0 |
| Clindamycin | — |
| Example 3 | |
| Erythromycin | — |
| Propylene glycol | 96.0 |
| ULTREZ 10 | 2.0 |
| Polyethylene glycol | — |
| Clindamycin | 1.0 |
| Example 4 | |
| Erythromycin | 2.0 |
| Propylene glycol | 96.0 |
| ULTREZ 10 | 1.0 |
| SEPIGEL 305 | 1.0 |
| Example 5 | |
| Propylene glycol | 66.5 |
| Vitamin A 50% | 2.0 |
| Carbopol | 1.5 |
| Silicone | 10.0 |
| C₁₂₋₁₅ Alkyl Benzoate | 5.0 |
| Starch | 15.0 |
| Example 6 | |
| Propylene glycol | 59.50 |
| Ascorbic Acid | 8.00 |
| Carbopol | 1.50 |
| Silicon | 10.0 |
| C₁₂₋₁₅ Alkyl Benzoate | 5.0 |
| Starch | 15.0 |
| Licorice extract | 1.0 |
| Example 7 | |
| Propylene glycol | 58.5 |
| Ascorbic Acid | 8.0 |
| Vitamin A 50% | 2.0 |
| Carbopol | 1.5 |
| Silicone | 10.0 |
| C₁₂₋₁₅ Alkyl Benzoate | 5.0 |
| Starch | 15.0 |
| Example 8 | |
| Skin whitening gel | |
| Propylene glycol | 54.5 |
| Ascorbic Acid | 8.0 |
| Vitamin A 50% | 2.0 |
| Carbopol | 1.5 |
| Silicone | 10.0 |
| C₁₂₋₁₅ Alkyl Benzoate | 5.0 |
| Hydroquinone | 4.0 |
| Starch | 15.0 |
| Example 9 | |
| Self Tanning gel | |
| Propylene glycol | 67.5 |
| Dihydroxy acetone | 7.0 |
| Carbopol | 1.5 |
| Silicone | 10.0 |
| C₁₂₋₁₅ Alkyl Benzoate | 5.0 |
| Hydroquinone | 4.0 |
| Starch | 15.0 |
| Example 10 | |
| Propylene glycol | 72.5 |
| Ascorbic Acid | 5.0 |
| Green Tea Extract | 1.0 |
| Carbopol | 1.5 |
| Silicone | 5.0 |
| Starch | 15.0 |

### EXAMPLES 11-14

The following exemplary benzoyl peroxide-containing formulations are suitable for use as the second composition to be dispensed simultaneously with any of the anhydrous formulations of Examples 1-10.

| Example 11 -Gel Composition | |
|---|---|
| Water | 56.4 |
| Glycerine | 5.0 |
| SEPIGEL 305 | 2.0 |
| Sodium Hydroxide | 1.60 |
| Steareth S-20 | 2.0 |
| Steareth S-2 | 2.0 |
| Cetyl Stearyl Alcohol | 3.0 |
| Silicone Cupoydoyl | 5.0 |
| Lucidol 75% (Benzoyl Peroxide) | 16.0 |
| C₁₂₋₁₅ Benzoate Ester | 7.00 |
| Example 12 | |
| Clear gel | |
| Water | 89.5 |
| Carbopol | 2.0 |
| Alcohol | 8.0 |
| Triethanolamine | 0.5 |
| Example 13 | |
| Oil in water Nonionic Emulsion | |
| Water | 86.35 |
| Glycerine | 3.00 |
| Methyl Paraben | 0.20 |
| Disodium EDTA | 0.05 |
| Steareth S-20 | 0.50 |
| Silicone | 1.0 |
| Propyl Paraben | 0.10 |
| Cetyl Stearyl Alcohol | 4.20 |
| Petrolatum | 3.0 |
| Perfume oil | 0.3 |
| Example 14 | |
| Oil in water anionic emulsion | |
| Water | 79.55 |
| Glycerine | 5.00 |
| Methyl Paraben | 0.18 |
| Disodium EDTA | 0.05 |
| Veegum | 0.40 |
| Polydecene | 3.50 |
| Sesame oil | 2.50 |
| Silicone | 0.20 |
| Glycerol Monostearate | 1.00 |
| Stearic Acid | 2.50 |
| Cetyl Alcohol | 0.70 |
| Propyl Paraben | 0.12 |
| Carbopol | 3.0 |
| Fragrance | 0.3 |

It will be understood that various modifications may be made to the embodiments disclosed herein. Therefore, the above description should not be construed as limiting, but merely as exemplifications of preferred embodiments. Those skilled in art will envision other modifications within the scope and spirit of the claims appended hereto.

We claim:

1. An apparatus comprising:
a first chamber containing multiple doses of a first composition, the first composition being substantially anhydrous and comprising a first active ingredient that is unstable in the presence of water and provides a benefit to the skin of a user;
a second chamber containing a second composition, the second composition containing water; and
one or more outlets for dispensing the first and second compositions.

2. An apparatus as in claim 1 wherein the first composition comprises an active ingredient selected from the group consisting of antibiotics, ascorbic acid, ascorbic acid derivatives, retinoids, hydroquinone, dihydroxyacetone, licorice extract and green tea extract.

3. An apparatus as in claim 1 wherein the first active ingredient is an antibiotic.

4. An apparatus as in claim 1 wherein the first active ingredient is selected from the group consisting of ascorbic acid and ascorbic acid derivatives.

5. An apparatus as in claim 3 wherein the antibiotic is selected from the group consisting of erythromycin, clindamycin, tetracycline, derivatives of erythromycin, clindamycin or tetracycline and pharmaceutically acceptable salts of erythromycin, clindamycin or tetracycline.

6. An apparatus as in claim 1 wherein the second composition contains an active ingredient.

7. An apparatus as in claim 6 wherein the second composition contains benzoyl peroxide.

8. An apparatus as in claim 7 wherein benzoyl peroxide comprises from about 0.1 to about 25 weight percent of the second composition.

9. An apparatus as in claim 1 wherein the first composition comprises a retinoid.

10. An apparatus as in claim 9 wherein the retinoid is selected from the group consisting of retinol, retinoic acid, retinyl palmitate, retinyl propionate, retinyl acetate and synthetic retinoid mimetics.

11. An apparatus as in claim 1 wherein the first composition comprises
i) a polar solvent;
ii) said first active ingredient; and
iii) a thickening agent in an amount sufficient to impart to the first composition a viscosity of at least 1000 cenetipoise measured at room temperature.

12. An apparatus as in claim 10 wherein the first composition contains a polar solvent selected from the group consisting of polyols and polyhydric alcohols.

13. An apparatus as in claim 1 further comprising pump means for moving the first and second compositions out of the first and second chambers.

14. A method of providing a beneficial effect to the skin of a user comprising
simultaneously dispensing a first composition and a second composition from first and second chambers, respectively,
the first being substantially anhydrous and comprising a first active ingredient that is unstable in the presence of water and provides a benefit to the skin of a user, the second composition comprising water and optionally a second active ingredient; and
contacting the first composition and second composition with the skin of a user.

15. An method as in claim 14 wherein the first composition comprises an active ingredient selected from the group consisting of antibiotics, ascorbic acid, ascorbic acid derivatives, retinoids, hydroquinone, dihydroxyacetone, licorice extract and green tea extract.

16. A method as in claim 14 wherein the first active ingredient is an antibiotic.

17. A method as in claim 14 wherein the first active ingredient is selected from the group consisting of ascorbic acid and ascorbic acid derivatives.

18. A method as in claim 17 wherein the antibiotic is selected from the group consisting of erythromycin, clindamycin, tetracycline, derivatives of erythromycin, clindamycin or tetracycline and pharmaceutically acceptable salts of erythromycin, clindamycin or tetracycline.

19. A method as in claim 14 wherein the second composition contains an active ingredient.

20. A method as in claim 19 wherein the second composition contains benzoyl peroxide.

21. A method as in claim 20 wherein benzoyl peroxide comprises from about 0.1 to about 25 weight percent of the second composition.

22. A method as in claim 14 wherein the first composition comprises a retinoid.

23. A method as in claim 22 wherein the retinoid is selected from the group consisting of retinol, retinoic acid, retinyl palmitate, retinyl propionate, retinyl acetate and synthetic retinoid mimetics.
